(19) United States
(12) Patent Application Publication
(10) Pub. No.: US 2015/0011734 A1
(43) Pub. Date: Jan. 8, 2015

(54) PROTEIN AND GENE RELATED WITH BASAL THERMOTOLERANCE OF PLANTS

(71) Applicant: National Central University, Taoyuan (TW)

(72) Inventors: Shaw-Jye Wu, New Taipei City (TW); Lian-Chin Wang, Taipei City (TW); Jia-Rong Wu, Kaohsiung City (TW)

(73) Assignee: National Central University, Taoyuan (TW)

(21) Appl. No.: 14/093,501

(22) Filed: Dec. 1, 2013

(30) Foreign Application Priority Data
Jul. 5, 2013 (TW) 102124196

(51) Int. Cl. C07K 14/415

(52) U.S. Cl. CPC C07K 14/415
USPC 530/370; 536/23.6

(57) ABSTRACT

An isolated protein having SEQ ID NO:1 and an isolated gene encoding the protein are provided. The protein is related with basal thermotolerance of plants having the protein.

### CROSS-REFERENCE TO RELATED APPLICATION

This application claims the priority benefit of Taiwan application serial no. 102124196, filed on Jul. 5, 2013. The entirety of the above-mentioned patent application is hereby incorporated by reference herein and made a part of this specification.

### BACKGROUND OF THE INVENTION

1. Field of the Invention

The invention relates to a protein and a gene, and more particularly, to a protein and a gene related with basal thermotolerance of plants.

2. Description of Related Art

Greenhouse effect caused a rise in temperature around the globe, and the warming climate directly affects problems related to ecology and food. Moreover, temperature variations directly affect plant growth and development, and may cause damage to the plants, and even cause the plants to die. Past research relating to plants resisting heat stress mainly focuses on heat shock protein (HSP) and the mechanism of acquired thermotolerance of plants. Acquired thermotolerance refers to the ability of plants to survive when faced with lethal heat stress after being acclimatized by non-lethal high temperature. In general, in addition to short-term heat stress, sustained heat stress also exists in the environment. However, complete research is lacking regarding how plants survive under sustained heat stress and the regulatory mechanism (mechanism of basal thermotolerance) of plants facing such stress. Therefore, understanding the mechanism of how plants resist sustained heat stress can benefit the improvement of crop varieties, thereby increasing crop yield and quality.

### SUMMARY OF THE INVENTION

The invention provides an isolated protein having SEQ ID NO:1 and an isolated gene encoding the protein. The isolated protein having SEQ ID NO:1 is related with basal thermotolerance of plants.

The invention provides an isolated protein having SEQ ID NO:1. The isolated protein having SEQ ID NO:1 is related with basal thermotolerance of plants having the protein.

The invention further provides an isolated gene encoding the protein. The protein is related with basal thermotolerance of plants having the protein.

In an embodiment of the invention, the plants include Arabidopsis.

In another embodiment of the invention, the gene has SEQ ID NO:2 or a degenerated sequence thereof.

In another embodiment of the invention, the gene is originated from Arabidopsis.

In each embodiment of the invention, the expression of the protein increases the tolerance of plants having the protein to high temperature in an environment.

In each embodiment of the invention, the protein has the ability to remodel the chromatin of plants having the protein.

Based on the above, the isolated protein of the invention has SEQ ID NO:1 and the protein is related with basal thermotolerance of plants having the protein. Therefore, the invention discloses a regulator molecule of plants for heat stress response, wherein the participating regulatory mechanism thereof is relatively important to basal thermotolerance of plants.

To make the above features and advantages of the invention more comprehensible, several embodiments accompanied with drawings are described in detail as follows.

### BRIEF DESCRIPTION OF THE DRAWINGS

The patent or application file contains at least one drawing executed in color.

Copies of this patent or patent application publication with color drawing(s) will be provided by the Office upon request and payment of the necessary fee. The accompanying drawings are included to provide a further understanding of the invention, and are incorporated in and constitute a part of this specification. The drawings illustrate embodiments of the invention and, together with the description, serve to explain the principles of the invention.

FIG. 1 shows the analysis results of subsequent growth and survival rate of Arabidopsis wild type seedlings and Arabidopsis mutant seedlings after various high temperature treatments.

FIG. 2 are micrographs of staining nuclei of protoplasts from Arabidopsis expressing fluorescently tagged HIT4 protein, and at the same time counterstaining DNA in the nuclei with DAPI.

FIG. 3 shows the analysis results of conformational change of chromocentres of nuclei from Arabidopsis wild type seedlings and mutant seedlings after various high temperature treatments.

### DESCRIPTION OF THE EMBODIMENTS

The invention provides an isolated protein having SEQ ID NO:1. The isolated protein having SEQ ID NO:1 is related with basal thermotolerance of plants having the protein. More specifically, the expression of the protein can increase the tolerance of plants having the protein to high temperature in an environment. Moreover, the protein has the ability to remodel the chromatin of plants having the protein. The plants are, for instance, Arabidopsis, but the invention is not limited thereto. In other embodiments, the protein can also be isolated from tomatoes or other plants having the protein.

The invention further provides an isolated gene encoding the protein. The nucleic acid sequence of the complementary DNA (cDNA) of the gene is, for instance, SEQ ID NO:2 or a degenerated sequence thereof. The gene is originated from, for instance, Arabidopsis, but the invention is not limited thereto. In other embodiments, the gene can also be originated from tomatoes or other plants having the gene.

In the following, the protein and the gene of the invention are investigated in two parts to disclose the molecular function and physiological function of each thereof, wherein the first part is the construction of Arabidopsis mutants with loss of basal thermotolerance and the other is physiological tests of the Arabidopsis mutants. It should be mentioned that, experiments relating to the construction of Arabidopsis mutant hit4-1 and physiological tests can be performed by those skilled in the art using known operating techniques of molecular biology. Alternatively, those skilled in the art can perform the experiments through the contents of the research paper titled

“Arabidopsis HIT4 encodes a novel chromocentre-localized protein involved in the heat reactivation of transcriptionally silent loci and is essential for heat tolerance in plants” of the invention claiming novelty priority. The research paper was published in the Journal of Experimental Botany in 2013 and the entire content of the research paper is incorporated into the present specification with reference to its entire content. The research paper is therefore not repeated herein.

[Construction of Arabidopsis Mutants with Loss of Basal Thermotolerance]

In general, since Arabidopsis has such advantages as short life cycle, small size, and produces a large number of offspring, Arabidopsis has been widely used in the experiments of genetics and molecular biology, and therefore Arabidopsis is selected for use in the present experiment.

To find the genetic determinants responsible for the tolerance of heat stress in plants, the present experiment uses the strategy of forward genetics and uses the chemical mutagen ethyl methanesulphonate (EMS) to apply point mutations to Arabidopsis wild type to screen for a mutant (hereinafter Arabidopsis mutant hit4-1 (heat-intolerant 4-1)) that loses thermotolerance to sustained high temperature. The Arabidopsis mutant hit4-1 loses basal thermotolerance but still retains the acquired thermotolerance.

Moreover, in the present experiment, the locus of the mutation site is mapped through map-based cloning (MBC) to confirm the heat sensitivity of the Arabidopsis mutant hit4-1 is caused by the point mutation of the gene labeled At5g10010 on chromosome 5 of the Arabidopsis. More specifically, a single point mutation in which nucleotide C is replaced by A occurs at the 680th by in the sixth exon of the At5g10010 gene on chromosome 5 of the Arabidopsis mutant hit4-1, causing the 227th amino acid of the protein encoded by the Arabidopsis mutant hit4-1 to transform from serine to tyrosine. As a result, the Arabidopsis mutant hit4-1 loses thermotolerance to sustained high temperature. In the following, the gene labeled as At5g10010 is referred to as HIT4 gene and the protein transcribed by the HIT4 gene is referred to as HIT4 protein, wherein the HIT4 gene is an isolated gene having SEQ ID NO:2 (cDNA sequence) and the HIT4 protein is an isolated protein having SEQ ID NO:1 (amino acid sequence).

[Physiological Tests of Arabidopsis Mutants]

Since the HIT4 gene is still not researched to this day, the molecular function of the HIT4 protein and the physiological effects thereof to cells are completely unknown. In the following, physiological tests such as tolerance of Arabidopsis mutant hit4-1 to high temperature in an environment are explained through experimental examples to further understand the cellular mechanism and applications relating to the HIT4 protein.

### EXPERIMENTAL EXAMPLE 1

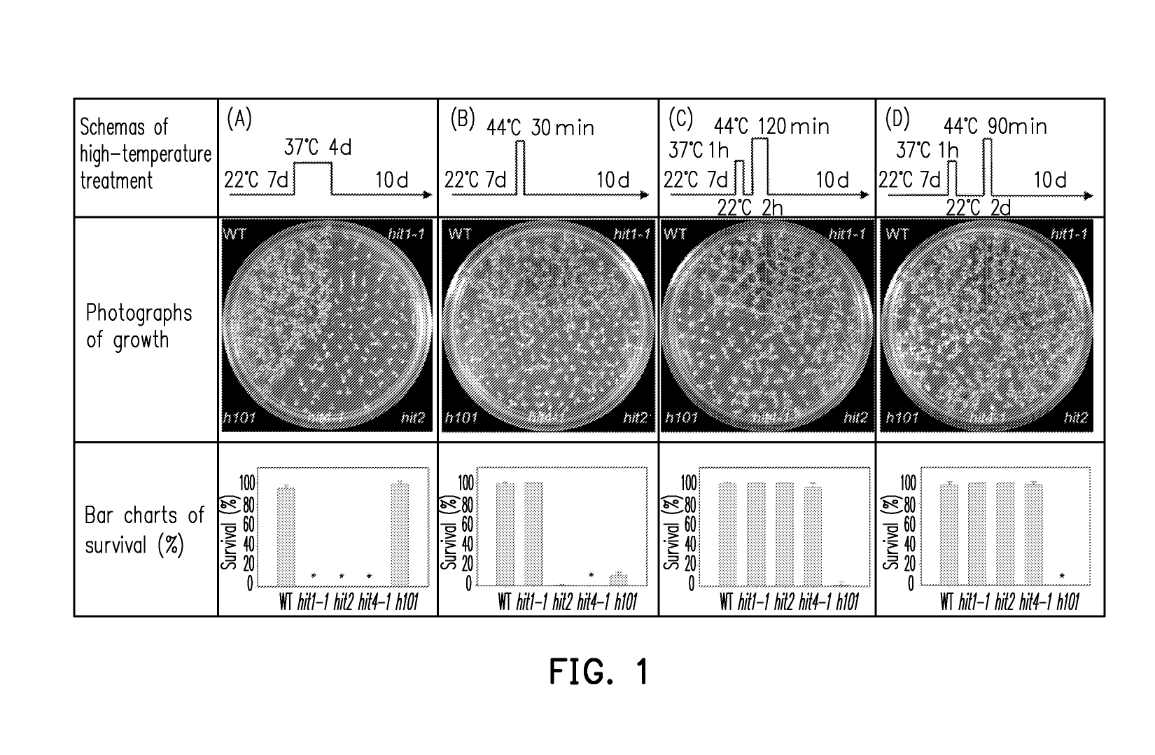

FIG. 1 shows the analysis results of subsequent growth and survival rate of Arabidopsis wild type seedlings and Arabidopsis mutant seedlings after various high temperature treatments. In FIG. 1, the experimental group is Arabidopsis mutant hit4-1 seedlings and the control group includes Arabidopsis wild type (abbreviated as WT in figures) and Arabidopsis mutants hit1-1, hit2, and h101 (hsp101 (heat shock protein 101)) seedlings, wherein the Arabidopsis mutants hit1-1, hit2, and h101 are known mutants with loss of tolerance to sustained heat stress (hit-1 and hit-2), sudden heat shock (hit2 and h101), or acquired thermotolerance (h101).

More specifically, (A), (B), (C), and (D) of FIG. 1 are respectively different high temperature treatment methods (as shown in the sketches) of 7-day-old Arabidopsis wild type seedlings and Arabidopsis mutant seedlings, wherein the 7-day-old seedlings are seedlings incubated at a constant temperature of 22° C. for 7 days. The high temperature treatment method of (A) of FIG. 1 is a long-term heat treatment at 37° C. for 4 days. The high temperature treatment method of (B) of FIG. 1 is a sudden heat shock treatment at 44° C. for 30 minutes. The high temperature treatment method of (C) of FIG. 1 includes first pre-acclimating at 37° C. for 1 hour, then incubating at 22° C. for 2 hours, and then applying a sudden heat shock treatment at 44° C. for 120 minutes. The high temperature treatment method of (D) of FIG. 1 includes first pre-acclimating at 37° C. for 1 hour, then incubating at 22° C. for 2 days, and then applying a sudden heat shock treatment at 44° C. for 90 minutes.

FIG. 1 also shows analysis results of subsequent growth (under the growth conditions of incubating at a constant temperature of 22° C. for 10 days for seedlings to recover) and survival rates, wherein the growth and the survival rates are respectively represented by photographs and bar charts. The Arabidopsis wild type seedlings and the Arabidopsis mutant seedlings are incubated in a solid medium and maintained under the growth condition of a 16-hour light/8-hour dark cycle, wherein the light intensity is 100 μmol m⁻²s⁻¹. Moreover, the survival rate (%) is calculated through the number of plants that continued to survive (green leaf growth) after the high temperature treatment, wherein each data is the average value of 3 repeated experiments and a testing rod represents the standard deviations of all of the experiments. The “ *” symbol in the figure represents the survival rate of the plants is zero.

(A) of FIG. 1 shows the experimental results of sustained heat stress. It can be known from the growth and survival rates of incubating at 22° C. for 10 days after a long-term heat treatment at 37° C. for 4 days that the Arabidopsis wild type and Arabidopsis mutant h101 (with loss of tolerance to sudden heat shock and acquired thermotolerance) seedlings are unaffected and continue to grow, but the Arabidopsis mutant hit4-1 seedlings are completely bleached to death. Therefore, the Arabidopsis mutant hit4-1 with single point mutation loses thermotolerance to sustained heat stress. In other words, basal thermotolerance of the Arabidopsis mutant hit4-1 with single point mutation is lost. Moreover, in the control group, it is known that the Arabidopsis mutants hit1-1 and hit2 with loss of tolerance to sustained heat stress are also completely bleached to death. The results are consistent with the Arabidopsis mutant hit4-1, further confirming the Arabidopsis mutant hit4-1 lost basal thermotolerance.

(B) of FIG. 1 shows the test results of sudden heat shock. It can be known from the growth and survival rates of incubating at 22° C. for 10 days after sudden heat shock at 44° C. for 30 minutes that the Arabidopsis wild type and Arabidopsis mutant hit1-1 (with loss of tolerance to sustained heat stress) seedlings are unaffected and continue to grow, but the Arabidopsis mutant hit4-1 seedlings are completely bleached to death. Therefore, the Arabidopsis mutant hit4-1 with single point mutation loses tolerance to sudden heat shock. Moreover, in the control group, it is known that the Arabidopsis mutants hit2 and h101 with loss of tolerance to sudden heat shock are also completely bleached to death. The results are consistent with the Arabidopsis mutant hit4-1, further confirming the Arabidopsis mutant hit4-1 loses thermotolerance to sudden heat shock.

(C) and (D) of FIG. 1 show the test results of acquired thermotolerance, wherein (C) of FIG. 1 includes pre-acclimating at 37° C. for 1 hour, then incubating at 22° C. for 2 hours, and then applying a sudden heat shock treatment at 44° C. for 120 minutes, and (D) of FIG. 1 includes pre-acclimating at 37° C. for 1 hour, then incubating at 22° C. for 2 days, and then applying a sudden heat shock treatment at 44° C. for 90 minutes. It can be known from the growth and survival rates after incubating at 22° C. for 10 days that the Arabidopsis wild type seedlings and the seedlings of the Arabidopsis mutants hit1-1, hit2, and hit4-1 are unaffected and continue to grow. Therefore, the Arabidopsis mutant hit4-1 with single point mutation still has acquired thermotolerance. Moreover, in the control group, it is known that the Arabidopsis mutant h101 with loss of tolerance for acquired thermotolerance is completely bleached to death. The result is opposite to the Arabidopsis mutant hit4-1, further confirming the Arabidopsis mutant hit4-1 still retains the acquired thermotolerance.

As shown in (A), (B), (C), and (D) of FIG. 1, it can be known from the test results of the sustained heat stress, the sudden heat shock, and the acquired thermotolerance that the Arabidopsis mutant hit4-1 with single point mutation lost basal thermotolerance but still retained acquired thermotolerance. Therefore, the HIT4 gene and the HIT4 protein transcribed by the HIT4 gene are related with basal thermotolerance of plants.

### EXPERIMENTAL EXAMPLE 2

FIG. 2 shows micrographs of staining the nuclei of the protoplasts from Arabidopsis expressing fluorescently tagged HIT4 protein, and at the same time counterstaining DNA in the nuclei with DAPI (4′,6′-diamidino-2-phenylindole). Here, the fluorescently tagged HIT4 protein is referred to as GFP-HIT4 protein, wherein the GFP-HIT4 protein is a HIT4 protein tagged with green fluorescent protein formed through in frame fusion of the C-terminal of a green fluorescent protein (GFP) and the N-terminal of the HIT4 protein.

In the present experimental example, the active site of the HIT4 protein in the cells is observed using the protoplasts from Arabidopsis expressing the GFP-HIT4 protein. (A) of FIG. 2 is a micrograph of the nuclei of the protoplasts from Arabidopsis expressing the GFP-HIT4 protein observed under bright field, wherein the bar in the figure is 5 microns. (B) of FIG. 2 is a fluorescence micrograph of the nuclei of the protoplasts from Arabidopsis expressing the GFP-HIT4 protein. It is known that there are 10 chromosomes in the nuclei of the protoplasts from Arabidopsis, and therefore the green fluorescence signals (about 10 in number) emitted by the GFT-HIT4 protein in (B) of FIG. 2 may respectively represent the locations of chromocentres.

As shown in (C) of FIG. 2, to further confirm the GFP-HIT4 protein is mainly distributed on the chromocentre, the present experiment further uses DAPI staining for DNA to confirm the location of cell nucleus, wherein blue fluorescence signal represents the location of cell nucleus. Then, (B) of FIG. 2 and (C) of FIG. 2 are merged to be the fluorescence micrograph of (D) of FIG. 2. It can be seen from (D) of FIG. 2 that the green fluorescence signal (location of each of the GFP-HIT4 protein and the chromocentre) is overlapped with the blue fluorescence signal (location of cell nucleus). Therefore, it can be confirmed that the HIT4 protein in the cells is mainly distributed in the cell nucleus and distributed on the chromocentre formed by condensing heterochromatin.

### EXPERIMENTAL EXAMPLE 3

Recently, research has indicated that long-term heat stress causes the tightly folded structure of heterochromatin to disappear, and can activate the repeated sequence of transcriptional gene silencing (TGS). Moreover, experimental example 1 confirms the Arabidopsis mutant hit4-1 is a mutant with loss of thermotolerance and experimental example 2 confirms the HIT4 protein is in the chromocentre of the cell nucleus. Therefore, in the present experimental example, conformational changes to the chromocentre are observed through a DAPI stain and fluorescent in situ hybridization (FISH) to further understand the cellular mechanism involved with the HIT4 protein.

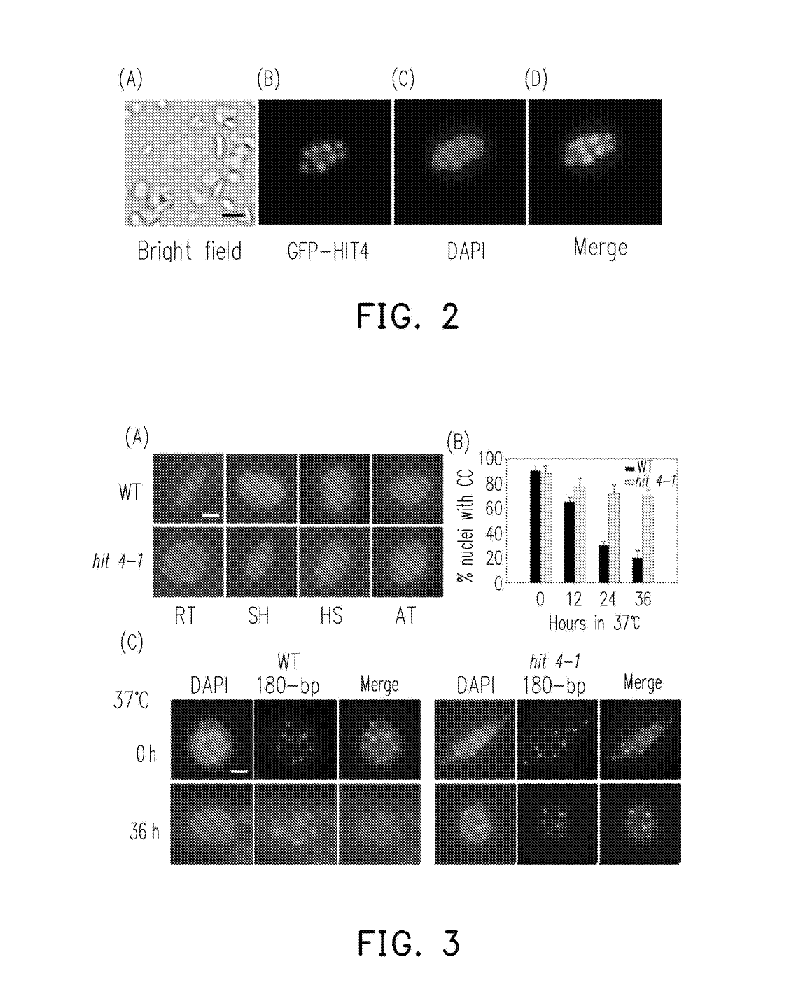

FIG. 3 shows the analysis results of conformational change of the chromocentres of the nuclei from the Arabidopsis wild type seedlings and the mutant hit4-1 seedlings after various high temperature treatments, wherein the bar in the figure is 5 microns. (A) of FIG. 3 is the analysis result of conformational changes of the chromocentres of the nuclei from the Arabidopsis wild type seedlings and the mutant hit4-1 seedlings observed through the DAPI stain after various high temperature treatments. In (A) of FIG. 3, RT (room temperature) represents the control group at room temperature (i.e. before high temperature treatment), SH (sustained heat stress) represents sustained high temperature treatment at 37° C. for 36 hours (basal thermotolerance test), HS (sudden heat shock) represents sudden heat shock treatment at 44° C. for 30 minutes, and AT (acquired thermotolerance) represents pre-acclimating at 37° C. for 1 hour, then incubating at 23° C. for 2 hours, and then applying a sudden heat shock treatment at 44° C. for 120 minutes (acquired thermotolerance test).

(B) of FIG. 3 shows the results of the quantitative analysis of nuclei with condensed chromocentres (hereinafter CC) in the Arabidopsis wild type and the mutant hit4-1 before and after heat stress at 37° C. (that is, including sustained high temperature treatment at 37° C. for 0, 12, 24, and 36 hours). In particular, nuclei with CC (%) is calculated through a number of 100 nuclei of each plant, wherein each value is the average value of 5 repeated experiments and a testing rod is used to show the standard deviations of all of the experiments.

It can be known from (A) and (B) of FIG. 3 that, before the heat stress at 37° C., the nuclei with CC in the Arabidopsis wild type and the mutant hit4-1 are both greater than 70%. However, after the heat stress at 37° C. for 36 hours, the nuclei with CC in the Arabidopsis wild type is significantly reduced to about 20%, but in contrast the nuclei with CC in the Arabidopsis mutant hit4-1 is still retained at about 70%. In other words, as shown in the basal thermotolerance test (SH) of (A) of FIG. 3, sustained high temperature treatment induced chromocentre decondensation in the Arabidopsis wild type. However, it does not occur in the Arabidopsis mutant hit4-1 with loss of basal thermotolerance. Moreover, as shown in the sudden heat shock test (HS) of (A) of FIG. 3, sudden heat shock treatment induced chromocentre decondensation in the Arabidopsis wild type. However, it does not occur in the Arabidopsis mutant hit4-1. Moreover, as shown in the acquired thermotolerance test (AT) of (A) of FIG. 3, the acquired thermotolerance test induced chromocentre decondensation in the Arabidopsis wild type. It is similar to the level of chromocentre decondensation induced by the Arabidopsis mutant hit4-1 with retained acquired thermotolerance. It can be known from the results of the tests that the reorganization of chromocentres is a key response in plants for heat tolerance.

(C) of FIG. 3 shows the analysis results of chromocentre decondensation of the nuclei from the Arabidopsis wild type seedlings and the mutant hit4-1 seedlings observed through the DAPI stain and fluorescent in situ hybridization (FISH) before and after heat stress at 37° C. (that is, including sustained high temperature treatment at 37° C. for 0 and 36 hours). In (C) of FIG. 3, the fluorescent in situ hybridization (FISH) confirms the number and conformational changes of chromocentres through a centromeric 180-bp probe of fluorescent labeling (such as green fluorescent labeling), wherein the 180-bp is a short single-stranded DNA sequence derived from the repeated sequence in each chromosome.

The DAPI staining test of the Arabidopsis wild type and the mutant hit4-1 is the same as the before high temperature treatment test (RT) and the basal thermotolerance test (SH) of (A) of FIG. 3 and is not repeated herein. To further observe chromocentre decondensation of the nuclei, the present experimental example further uses the 180-bp for counterstain to confirm the number and conformational changes of the chromosomes. The 180-bp (FISH test) of (C) of FIG. 3 is a fluorescence micrograph of the chromosomes (10 in number) of the nuclei from Arabidopsis seedlings. As shown in (C) of FIG. 3, the green fluorescence signal represents the number and conformational changes of the chromosomes. Then, the fluorescence micrograph of each of the DAPI stain and the 180-bp are merged. Since the blue fluorescence signal (location of cell nucleus) and the green fluorescence signal (number and conformational changes of chromosomes) are overlapped, it can be confirmed that the chromocentre decondensation and TGS activation of the Arabidopsis mutant hit4-1 in sustained heat stress are not significant. In other words, the Arabidopsis mutant hit4-1 with loss of basal thermotolerance does not induce chromocentre decondensation, and therefore the TGS is not activated.

It can be known from the experimental results that the invention discloses the HIT4 protein is a regulator molecule of plants for heat stress response, wherein the HIT4 protein has the function of chromatin remodeling. This function can promote activation of the TGS. Moreover, the regulatory mechanism through the HIT4 protein is essential to basal thermotolerance of plants. Therefore, the cellular mechanism involved with the HIT4 protein can be applied to such fields as crop improvement to increase crop yield and quality.

Based on the above, in the protein and the gene encoding the protein of the invention, the protein has SEQ ID NO:1 and the protein is related with basal thermotolerance of plants having the protein. The experimental examples of the invention disclose the protein is a regulator molecule of plants for heat stress response, wherein the protein has the function of chromatin remodeling (that is, chromocentre decondensation) to promote activation of the TGS. Moreover, the regulatory mechanism is essential to basal thermotolerance of plants. Therefore, the cellular mechanism involved with the protein can be widely applied to such fields as crop improvement to develop crops that can, for instance, resist heat stress, and thereby increase crop yield and quality. Moreover, the gene may further be applied to, for instance, biomedical and pharmaceutical research and commercial fields in the future.

Although the invention has been described with reference to the above embodiments, it will be apparent to one of the ordinary skill in the art that modifications to the described embodiments may be made without departing from the spirit of the invention. Accordingly, the scope of the invention is defined by the attached claims not by the above detailed descriptions.

What is claimed is:

1. An isolated protein having SEQ ID NO:1, wherein the protein is related with basal thermotolerance of plants having the protein.

2. The isolated protein having SEQ ID NO:1 of claim 1, wherein expression of the protein can increase a tolerance of the plants having the protein to high temperature in an environment.

3. The isolated protein having SEQ ID NO:1 of claim 1, wherein the protein has an ability to remodel a chromatin of the plants having the protein.

4. The isolated protein having SEQ ID NO:1 of claim 1, wherein the plants comprise Arabidopsis.

5. An isolated gene encoding the isolated protein having SEQ ID NO:1 of claim 1, wherein the protein is related with basal thermotolerance of plants having the protein.

6. The isolated gene of claim 5, wherein the gene has SEQ ID NO:2 or a degenerated sequence thereof.

7. The isolated gene of claim 5, wherein expression of the protein increases a tolerance of the plants having the protein to high temperature in an environment.

8. The isolated gene of claim 5, wherein the protein has an ability to remodel a chromatin of the plants having the protein.

9. The isolated gene of claim 5, wherein the gene is originated from Arabidopsis.


SEQUENCE LISTING (2 sequences)

SEQ ID NO: 1 - protein, 434 aa, Artificial Sequence
  mutant
MKKGAKRKGVSKAGRKAAVAETQNDEVIEETTKTTQEESQQHEEEVVDEVKENGEEEEAKGDQEEEEDAKPDSLEEDEENQEDEVKAEEVKEEVEKKPVARRGGKRKRATKKDTEIKDEKKPVPKAKKPRAAKVKEEPVYFEEKRSLEDLWKVAFPVGTEWDQLDALYEFNWDFQNLEEALEEGGKLYGKKVYVFGCTEPQLVPYKGANKIVHVPAVVVIESPFPPSDKIGITSVQREVEEIIPMKKMKMDWLPYIPIEKRDRQVDKMNSQIFTLGCTQRRSALRHMKEDQLKKFEYCLPYFYQPFKEDELEQSTEVQIMFPSEPPVVCEFDWEFDELQEFVDKLVEEEALPAEQADEFKEYVKEQVRAAKKANREAKDARKKAIEEMSEDTKQAFQKMKFYKFYPQPSPDTPDVSGVQSPFINRYYGKAHEVL

SEQ ID NO: 2 - DNA, 1305 bp, Artificial Sequence
  mutant
atgaagaaaggagcgaagagaaagggtgtttcgaaagcaggtcgcaaagctgctgttgcggagactcagaacgatgaggtgatagaggagacgacgaagacgacgcaggaggagagtcaacagcatgaggaagaagtcgtcgacgaggtgaaggagaatggggaagaagaggaggctaagggagatcaagaggaagaagaggatgcgaaacctgattccttagaggaggatgaggagaatcaggaagatgaggtcaaagctgaggaagtaaaggaagaagttgagaagaaacctgttgctcgtcgtggtggtaaacgaaagagggctacgaagaaggatactgagattaaagatgagaagaaacctgttccgaaagctaagaaaccgagagcggccaaagttaaggaagagcctgtctacttcgaggagaagcgtagtctggaggatttgtggaaggttgcatttccagtgggaactgagtgggatcaattagatgcactttatgaattcaattgggatttccaaaatcttgaagaagcattggaggaaggaggaaagctctatgggaagaaggtttatgtctttggctgtacagaacctcaactagtcccctacaaaggcgcaaacaagattgtccatgtcccagctgttgttgttattgaatcaccctttccgccttctgataagataggaatcacatctgttcagagagaagtggaggaaatcattccaatgaagaagatgaaaatggactggcttccatacattccgattgagaaaagagatagacaagtggataagatgaattctcaaatttttactttgggttgtacgcagagaagatctgctctcagacatatgaaggaagatcaacttaagaagtttgagtattgccttccttatttctatcaaccctttaaggaagatgaacttgaacagagtactgaggtccaaataatgttcccctctgaacccccggttgtatgtgaatttgactgggagtttgatgaacttcaggaatttgtcgataaactggttgaagaggaagcattacctgctgaacaagcggatgaattcaaagaatatgtcaaagagcaagttcgagcagcaaagaaagcaaatcgagaggccaaagatgctcgaaagaaagcaatagaagaaatgagcgaagatactaagcaagcctttcaaaagatgaagttctacaaattctaccctcagccttcaccagatacaccagacgtctctggtgtccagtccccattcattaaccgatactatggaaaggctcatgaagtcctttga